(19) United States
(12) Patent Application Publication
(10) Pub. No.: US 2009/0291484 A1
(43) Pub. Date: Nov. 26, 2009

(54) Method to enhance production of paralytic shellfish toxins from dinoflagellate cultures

(76) Inventors: Erik Selander, Lysekil (SE); Henrik Pavia, Stromstad (SE)

Correspondence Address: LYNN E BARBER, P O BOX 16528, FORT WORTH, TX, 76162, US

(21) Appl. No.: 12/154,621

(22) Filed: May 23, 2008

(51) Int. Cl. C12N 1/38

(52) U.S. Cl. 435/244

(57) ABSTRACT

A simple solid phase extraction (SPE) method for continuous sequestering and concentration of waterborne cues from sea water conditioned with aquatic source organisms that induce toxin formation in dinoflagellates, and a method of increasing this toxin formation.

### BACKGROUND OF THE INVENTION

1. Field of the Invention

The present invention relates generally to enhanced (up to more than 25 fold) production of natural products from marine phytoplankton microorganisms such as dinoflagellates. More specifically this invention relates to a particular method using waterborne cues from grazers inducing increased paralytic shellfish toxins (PSTs) production in dinoflagellates.

PSTs are a group of highly potent neurotoxins. In the marine environment they are produced by unicellular plankton belonging to the group of dinoflagellates. The toxins may accumulate in shellfish and cause serious intoxications in human consumers. Apart from the neurotoxic properties, PSTs are useful in biomedical research and are currently evaluated for use in medical treatments, more specifically for local anesthesia during eye surgery and as spasmolyticum. PSTs are commercially produced for use as analytical standards in shellfish monitoring and related research programs and as a tool in biomedical research. Commercial production of PSTs rely on the harvesting and extraction of large scale plankton cultures. The proposed innovation introduces a new way to increase the yield in the production process. This innovation thus significantly facilitates the production process of PSTs.

2. Description of the Related Art

It can be appreciated that the main problem with conventional way of producing paralytic shellfish toxins is that they are extracted from large scale cultures of slow growing dinoflagellate cells. The culturing and low toxin content of the cells limits the yield of the process, and makes production exceedingly expensive. It has previously been well established that the production rate depends to some extent on the physical and chemical environment of the cultured organisms. The proposed way of enhancing the toxin production in dinoflagellate cultures is however new, and to our knowledge, the first and only report of grazer induced toxin formation in dinoflagellates. Previous work on PST production in dinoflagellates has revealed the optimal environmental conditions for toxin production in the producing cells, which is also important to optimize yields in cultures. The proposed mechanism is, however, also most efficient when conditions for toxin production are optimal. Hence, the effect of the present invention strongly enhances the toxin production rate regardless of whether previous known factors are optimized for production of toxins. This is to our knowledge the only known example of waterborne cues from grazers inducing increased toxin formation in marine phytoplankton. There are however reports of a similar mechanism in fresh water cyanobacteria, where the cell specific content of microcystein can be increased by waterborne cues from cladoceran grazers (1). This concerns a completely different toxin compared to the paralytic shellfish toxins covered in the current application.

Some of the key information in this patent application has been published by the applicants in a manuscript in a doctoral thesis (2), published the 25 of May 2007.

While the traditional way of producing PSTs may be suitable for the particular purpose which they address, they are not sufficiently effective for producing paralytic shellfish toxins cost efficiently and in sufficient amounts to meet the growing demands.

A primary object of the present invention is to provide a product that gives higher yields paralytic shellfish toxins from dinoflagellate sources

Another object of the present invention is to allow for easy and sterile additions of the yield increasing additives to dinoflagellate and other microalgal cultures

Other objects and advantages will be more fully apparent from the following disclosure and appended claims.

### SUMMARY OF THE INVENTION

The general purpose of the present invention is to provide new products based on the invention that make it possible to control the production rate of paralytic shellfish toxins from phytoplankton cultures such as dinoflagellates, previously limited by the low content of the desired compound per cells and the slow growth rate of producing cells. To attain this, the present invention generally comprises products containing chemical signals from the natural enemies of the dinoflagellates (and other phytoplankton) cells that induce these cells to produce up to more than 25 times more of the desired compound.

There has thus been outlined, rather broadly, the more important features of the invention in order that the detailed description thereof may be better understood, and in order that the present contribution to the art may be better appreciated. There are additional features of the invention that will be described hereinafter.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of the description and should not be regarded as limiting.

### BRIEF DESCRIPTION OF THE DRAWINGS

FIG. 1 shows a schematic figure of how water borne cues can be sequestered from purified seawater containing the intact source organisms. Water is pumped by a peristaltic pump 1 from the incubation vessel through a solid phase extraction column 2 that sequesters the cues. The water is that reintroduced to the incubation chamber through a larger solid phase extraction column 3 to eliminate contamination from pump or hosing.

FIG. 2 shows that active extracts obtained from copepod grazers can be administered to culture vessels dissolved in e.g. methanol. After the solvent has been evaporated, cultures of Alexandrium minutum kept in the vessels will produce significantly more paralytic shellfish toxins compared to controls, without any measurable reductions in growth rate. The activity of the compound persists with heating to 75 degrees for 20 minutes. Bars represent mean values of three replicates; error bars denote standard error of mean.

### DETAILED DESCRIPTION OF THE INVENTION AND PREFERRED EMBODIMENTS THEREOF

The production rate of paralytic shellfish toxins in marine dinoflagellates can be enhanced up to at least 28 times the normal production rate by chemical compounds (“signals” or “cues”) originating from the natural enemies of the Alexandrium cells (3). Furthermore, we have developed a method for the isolation of these signals from seawater containing the intact copepod Centropages typicus (preferably living) which has, so far, been the most efficient organism to induce production, and also from other species of copepod grazers.

### Detailed Method Description

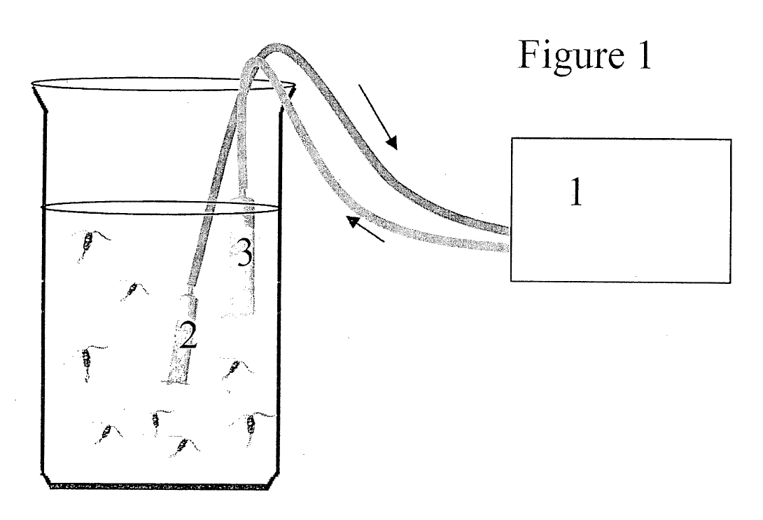

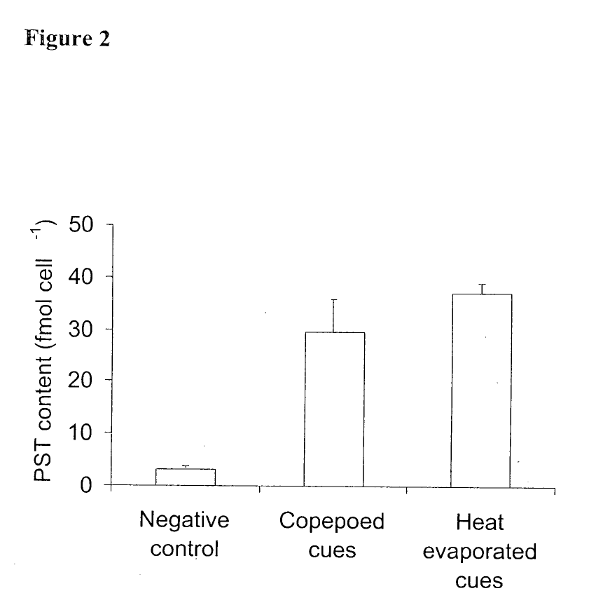

To obtain the chemical signals from copepod sources, the living intact source organisms, either obtained from the field or laboratory cultures, are placed in purified filtered seawater (FIG. 1). The source organisms need not be living for the method of the invention to function. The water is than continuously pumped by a peristaltic pump 1 through a solid phase extraction (SPE ENV+ Argonaut) column 2 and subsequently reintroduced into the vessel holding the source organism through a larger solid phase extraction column with the same packing to avoid contamination from pump and tubing 3. The SPE columns are than washed with distilled water to eliminate salts, and eluted with 100% methanol. The compound dissolved in methanol can than be added aseptically to culture vessels. The possibility to add the compound aseptically is important, because cultures are sensitive to contamination from other microorganisms or decaying copepods, and therefore safe, reproducible aseptic additions are necessary. When the methanol evaporates, the inside of the vessel is coated with the active substance and production is subsequently enhanced significantly in Alexandrium cells kept in the culture vessel. The signal compound(s) are not deactivated by evaporation in 75C for 20 minutes (FIG. 2). The kinetics of the induction depends on the life cycle of the responding dinoflagellates, but for paralytic shellfish toxin production in Alexandrium minutum, the effect develops after 1-2 days of incubation (2). It is probably possible to extract the compounds by extracting whole copepods as well and use the extracts as described above, only these extracts will be much more complex, containing all extractable compounds in the source copepods.

The calanoid copepods of the species Acartia tonsa, A. clausi and in particular Centropages typicus have been shown to induce PST production in Alexandrium minutum (3,4). Recent unpublished results have revealed that waterborne cues from the copepod Temora longicornis are also very efficient in inducing toxin formation in Alexandrium spp. It is likely that many other species of copepod species can be used in the same way, whereas others, e.g. Pseudocalanus sp. does not seem to have this effect (3).

The copepods should preferably be cultured in the laboratory to allow easy access to large numbers of animals of a single species. Copepod cultures can be reared on a diet of micro-alga, descriptions and methods are widely available in plankton and aquaculture literature.

Since the production of paralytic shellfish toxins is held back by the low yields from the producing cultures, this new product may be used to significantly increase yields and lower production costs for paralytic shellfish toxins produced from dinoflagellate cultures.

With respect to the above description then, it is to be realized that the optimum dimensional relationships for the parts of the invention, to include variations in size, materials, shape, form, function and manner of operation, assembly and use, are deemed readily apparent and obvious to one skilled in the art.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

### EXAMPLE 1

### Sequestering the Cues

Solid phase extraction (SPE) columns (Isolute ENV+ 50-500 mg columns, Argonaut Inc., Redwood City, Calif., USA) can be used to sequester the cue(s) from seawater conditioned with intact copepods. The ENV+ column packing has suitable properties to retain copepod derived signals from seawater and does not require conditioning with organic solvent before use which is considered an advantage because organic solvent from the conditioning may other ways have negative effects on the copepods.

The inducing cues can be continuously extracted from water containing copepod source organisms (FIG. 1) if water containing intact copepods, preferably living, is continuously pumped through a SPE column in a closed system (FIG. 1). It is likely that the activity can also be obtained from whole organism extracts from copepods, this will however result in a much more complex sample matrix, containing more compounds other than the actual inducing cue.

For the continuous cue extraction, filtered sea water (0.45 μm) purified through two 500 mg ENV+ columns was used to reduce contaminating compounds that are always present in filtered seawater. Approximately 1000-2000 copepods were suspended in 0.5-1 liter purified filtered seawater, but this should be scaled appropriately to fit the number of copepods and desired amount of cues. Water was than continuously drawn from the copepod containing vessel by a peristaltic pump (3 ml min⁻¹) through an ENV+ SPE column (50-300 mg) and reintroduced through a larger (500 mg) ENV+ column to avoid contamination from the pump or tubing. The volumes and amount of column packing depend on the number of copepods in the extraction vessel. Copepods, incubation water, and SPE columns should be exchanged daily to avoid starvation and starvation induced mortality. Columns can be stored frozen (−20 C) until use for enhancement of PST production.

### EXAMPLE 2

### Paralytic Shellfish Toxin Production Enhancement

The successful extraction of inducing cues was verified using a toxic strain of Alexandrium minutum, obtained from Göteborg University Marine Culture Collection, Gothenburg, Sweden (GUMACC #83 strain synonyms CCMP 113 Al and Al 1V). A. minutum was cultured in autoclaved K medium (5) at 18° C. with 150 μmol m⁻² s⁻¹ provided by fluorescent tubes,14:10h light:dark cycles. Experiments with other strains of A. minutum show similar results to the experiments with A. minutum GUMACC #83, and it can therefore be expected that other strains of Alexandrium spp. will respond in a similar way to copepod scents.

The sequestered compounds can be eluted from the SPE columns into a small volume of methanol (100%). The extract, now containing the active compound can be added to culture vessels and evaporated till dryness. Evaporation with centrifuge vacuum evaporation, a stream of nitrogen gas, or heating samples to 70 degrees C. in air, does not inhibit the activity of extracts from Centropages typicus (FIG. 2, and unpublished results). The culture vessels, now coated with the active substance can subsequently be supplied with A. minutum culture. Within 1 to 3 days incubation in 18° C., with 150 μmol m⁻² s⁻¹, 14:10h light:dark cycle, provided by fluorescent tubes, a strong increase in toxicity will develop in the culture. Cultured cells can be harvested by filtration or centrifugation. The paralytic shellfish toxins can be extracted from the harvested cells through repeated freeze thaw actions in e.g. 0.05M acetic acid followed by preparative chromatography, as known in the art and not described in detail here.

To control the cell specific toxicity of the A. minutum culture, a known number of cells can be filtered onto glass fiber filters (e.g. 25 mm Ø Munketell MGA, Munktell A B, Falun, Sweden), lyophilized and stored frozen until analysis, with e.g. high performance liquid chromatography with post column derivatization and fluorescence detection. The growth rate of the induced A. minutum cells has not been significantly different to that of control cultures in experiments, and the increase in toxicity will therefore directly translate into a corresponding increase in yield.

The dose dependence and optimal addition of active compound will need to be established for each particular production process individually using standard methodology as known in the art.

### REFERENCES

1) Jang M H, Ha K, Joo G J, Takamura N (2003) Toxin production of cyanobacteria is increased by exposure to zooplankton. Freshwater Biology 48:1540-1550
2) Selander E (2007) Chemical ecology of paralytic shellfish toxin producing dinoflagellates. In: Department of Marine Ecology, vol. Ph.D. Göteborg University, Strömstad, p 128
3) Bergkvist J, Selander E, Pavia H (2008) Induction of toxin production in dinoflagellates: the grazer makes a difference. Oecologia Accepted
4) Selander E, Thor P, Toth G B, Pavia H (2006) Copepods induce paralytic shellfish toxin production in marine dinoflagellates. Proceedings of the Royal Society of London Series B-Biological Sciences 273:1673-1680
5) Leftley J W, Keller D K, Selvin R C, Claus W, Guillard R R L (1987) Media for the culture of oceanic ultraphytoplankton. Journal of Phycology 23:633-638

What is claimed is:

1. A method of enhancing production of paralytic shellfish toxins from a dinoflagellate culture comprising providing a compound from seawater containing a natural enemy of the dinoflagellate culture and exposing the dinoflagellate culture to the compound isolated from seawater containing a natural enemy of the dinoflagellate culture.

2. The method of claim 1, wherein the natural enemy of the dinoflagellate culture is an intact copepod.

3. The method of claim 2, wherein the intact copepod comprises Centropages typicus.

4. The method of claim 1, wherein the dinoflagellate culture is a culture of Alexandrium minutum.

5. A product containing paralytic shellfish toxins produced by the method of claim 1.

6. The product of claim 5, wherein the natural enemy of the dinoflagellate culture is an intact copepod.

7. The product of claim 6, wherein the intact copepod comprises Centropages typicus.

8. The product of claim 5, wherein the dinoflagellate culture is a culture of Alexandrium minutum.

9. A method for inducing phytoplankton cells to increase production of paralytic shellfish toxins, comprising placing the phytoplankton cells in a vessel containing chemical compounds originating from an intact source organism.

10. The method of claim 9, wherein the phytoplankton cells are dinoflagellate cells.

11. The method of claim 10, wherein the dinoflagellate cells are cells of Alexandrium minutum.

12. The method of claim 10, wherein the intact source organism is a calanoid copepod.

13. The method of claim 10, wherein the intact source organism is selected from the group consisting of Acartia tonsa, Acartia clausi, Temora longicornis and Centropages typicus.

14. The method of claim 13, wherein the intact source organism comprises Centropages typicus.

15. The method of claim 10, wherein the inside of the vessel is coated with the chemical compounds.

16. The method of claim 10, further comprising, prior to placing the dinoflagellate cells in the vessel:
a) placing the intact source organism in purified filtered seawater;
b) pumping the seawater and source organism through a solid phase extraction column;
c) washing the solid phase extraction column with distilled water;
d) eluting the solid phase extraction column with methanol so that compounds from the source organism is dissolved in the methanol;
e) aseptically adding the methanol containing the dissolved compound to a culture vessel; and
f) evaporating the methanol, so that when the methanol evaporates the inside of the vessel is coated with the compound.

17. The method of claim 10, wherein the compounds are continuously extracted from seawater containing the intact source organisms, wherein the seawater has been purified and is then pumped continuously through a solid phase extraction column in a closed system.

18. The method of claim 17, wherein the dinoflagellate cells are cells of Alexandrium minutum and the intact source organism comprises Centropages typicus.

19. The method of claim 10, wherein the dinoflagellate cells are incubated 1-2 days in the vessel.
